(19) United States
(12) Patent Application Publication
(10) Pub. No.: US 2022/0162470 A1
(43) Pub. Date: May 26, 2022

(54) COATINGS

(71) Applicant: MicroVention, Inc., Aliso Viejo, CA (US)

(72) Inventors: Xinping Wu, Aliso Viejo, CA (US); Petr Vasek, Costa Mesa, CA (US); Steve Plotkin, Beaumont, CA (US); Gregory M. Cruise, Rancho Santa Margarita, CA (US)

(21) Appl. No.: 17/534,886

(22) Filed: Nov. 24, 2021

Related U.S. Application Data
(60) Provisional application No. 63/118,189, filed on Nov. 25, 2020.

(51) Int. Cl. C09D 133/06; C09D 133/14; C08J 7/04; C08F 220/28; B05D 7/02; B05D 7/00

(52) U.S. Cl. CPC C09D 133/066; C09D 133/14; C08J 7/042; C08F 220/281; B05D 7/02; C08J 2433/14; B05D 2201/02; B05D 2425/01; B05D 2425/02; B05D 2502/00; C08J 2327/06; B05D 7/54

(57) ABSTRACT

Described herein are surface-treated polymeric substrates. Also described herein are polymeric coatings, which may be used as surface treatments.

### RELATED APPLICATIONS

This application claims priority of U.S. Provisional Patent Application No. 63/118,189, filed Nov. 25, 2020, the entire content of which is hereby incorporated by reference.

### FIELD

Described herein are preparations of substrates with covalently bonded polymer coatings that can increase the blood compatibility of the substrate.

### SUMMARY

Described herein are polymeric substrates with surfaces that exhibit blood compatibility. The polymeric substrates can include surfaces that are covalently coupled to a polymer that increases the hemocompatibility of the substrate.

In one embodiment, the polymer substrate can include thermoplastic polyurethanes, thermoplastic elastomers, thermoset elastomers, polyamides, polyesters, polystyrenes, polyether ether ketones, polyethylene vinyl acetate, polyvinylidene fluoride, polypropylene, polyethylene, polyvinyl chloride, polycarbonate, and combinations thereof. The polymeric substrate may be any convenient geometry, including tubes, rods, sheets, or more complex shapes. In one embodiment, the polymeric substrate is a film.

In some embodiments, the polymer is prepared by polymerizing an alkoxyalkyl (meth)acrylate or derivatives thereof and a second monomer containing an amine, a carboxylic acid, or a hydroxyl group. In one embodiment, the second monomer is aminoethyl methacrylate, aminopropyl methacrylamide, combinations thereof, or derivatives thereof. In another embodiment, the second monomer is acrylic acid, methacrylic acid, combinations thereof, and derivatives thereof. In another embodiment, the second monomer is hydroxyethyl methacrylate, hydroxyethyl acrylate, hydroxypropyl acrylate, hydroxybutyl acrylate, combinations thereof, or derivatives thereof.

Alternatively, in some embodiments, the polymer is prepared by polymerizing tetrahydrofurfuryl acrylate or derivatives thereof and a second monomer containing an amine, a carboxylic acid, or a hydroxyl group. In one embodiment, the second monomer is aminoethyl methacrylate, aminopropyl methacrylamide, combinations thereof, or derivatives thereof. In another embodiment, the second monomer is acrylic acid, methacrylic acid, combinations thereof, and derivatives thereof. In another embodiment, the second monomer is hydroxyethyl methacrylate, hydroxyethyl acrylate, hydroxypropyl acrylate, hydroxybutyl acrylate, combinations thereof, or derivatives thereof.

Following polymerization, reactive groups, preferably acrylates and/or methacrylates, are added to the copolymer via hydroxyl, amine, and/or carboxylic acid groups located in the monomers. Derivatization compounds can include, but are not limited to 2-isocyanatoethyl acrylate, 2-isocyanatoethyl methacrylate, N-hydroxysuccinimide ester of acrylic acid, N-hydroxysuccinimide ester of methacrylic acid, hetero-bifunctional poly(ethylene glycol) with acrylate and isocyanate groups, combinations thereof, and derivatives thereof.

In some embodiments, surface-treated substrates are described. These surface-treated substrates can include a substrate comprising a surface, and a least one copolymer. In some embodiments, the surface is covalently coupled to the copolymer, wherein the copolymer is prepared from the free radical polymerization of an alkoxyalkyl (meth)acrylate and at least one monomer containing amine, carboxylic acid, or hydroxyl functionality. In some embodiments, the surface is treated with a first copolymer (a base-coat) and subsequently treated with a second copolymer (a top-coat).

In some embodiments, the substrate includes thermoplastic polyurethanes, thermoplastic elastomers, thermoset elastomers, polyamides, polyesters, polystyrenes, polyether ether ketones, polyethylene vinyl acetate, polyvinylidene fluoride, polypropylene, polyethylene, polyvinyl chloride, polycarbonate, and combinations thereof.

In other embodiments, the at least one copolymer is further modified to contain a plurality of reactive moieties. In some embodiments, the plurality of reactive moieties is a plurality of acrylates.

In some embodiments, provided herein are plastic surfaces covalently linked to a base-coating of acrylated poly(THF-acrylate)-co-poly(APMA), which base-coating is subsequently covalently linked to a top-coating of acrylated poly(MEA)-co-poly(APMA).

### DETAILED DESCRIPTION

Described herein are polymeric substrates having a surface covalently coupled to polymers that can increase the hemocompatibility of the substrate.

Polymeric substrates are used in a variety of biomedical applications. In some applications, improved hemocompatibility of these substrates is desired. Some coatings can include phosphorylchloline, heparin, and poly(methoxyethyl acrylate). Many other molecules have been evaluated for coating of blood contacting, polymeric and metallic medical devices. However, a satisfactory, durable coating has not been found.

This disclosure details polymeric substrates with a surface treatment to increase hemocompatibility. One component of hemocompatibility is durability. For example, the surface treatments described herein have improved durability as compared to non-covalently linked surface treatments. The improvement in durability may manifest as a longer period of time before the surface treatments begin to degrade or separate from the treated surface. Thus, in some embodiments, the surface treatments described herein remain attached to the surface and intact for a period of time of at least 3 hours when in contact with blood. In some embodiments, the period of time is at least 4 hours, at least 5 hours, or at least 6 hours. In some embodiments, the period of time is at least 12 hours, or at least 24 hours. In some embodiments, the surface treatments described herein remain attached to the surface and intact for a period of time of at least one week, at least one month, or at least one year when in contact with water.

Application methods using the polymer described herein are also described. Although the present disclosure discusses medical devices, the polymer and methods described herein are applicable to any polymeric substrate in need of treatment.

The substrate for the coating may be any suitable polymeric material. Polymeric substrates can include, but are not limited to thermoplastic polyurethanes, thermoplastic elastomers, thermoset elastomers, polyamides, polyesters, polystyrenes, polyether ether ketones, polyethylene vinyl acetate, polyvinylidene fluoride, polypropylene, polyethylene, polyvinyl chloride, polycarbonate, and combinations thereof. A preferred polymeric substrate is polyvinyl chloride film.

Polymers used for coating can exhibit reduced thrombogenicity and or increased hemocompatibility. The polymers can be prepared by polymerizing at least two or more monomers. The first monomer can be represented by the formula

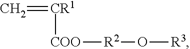

where R¹ is a hydrogen atom or methyl group,

R² is an alkylene group with 1 to 4 carbons, and

R³ is an alkylene group with 1 to 4 carbons.

In one embodiment, a first monomer is methoxyethyl acrylate wherein R¹ is a hydrogen atom, R² is an ethyl group, and R³ is a methyl group. In another embodiment, a first monomer is methoxyethyl methacrylate wherein R¹ is a methyl group, R² is an ethyl group, and R³ is a methyl group.

The first monomer can also include

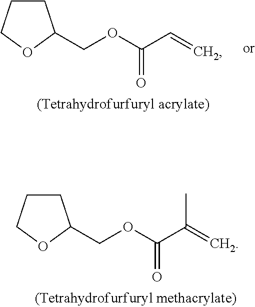

The second monomer can include a polymerizable acrylate or methacrylate as well as an amine, carboxylic acid, or hydroxyl group.

Monomers containing amines can include, but are not limited to 3-aminopropyl methacrylamide, 2-aminoethyl methacrylate, N-(3-methylpyridine)acrylamide, 2-(N,N-dimethylamino)ethyl methacrylate, 2-(N,N-dimethylamino)ethyl acrylate, 2-(tert-butylamino)ethyl methacrylate, methacryloyl-L-lysine, N-(2-(4-aminophenyl)ethyl)acrylamide, N-(4-aminobenzyl)acrylamide, and N-(2-(4-imidazolyl)ethyl)acrylamide, derivatives thereof, and combinations thereof. Monomers containing amines can also include aminopropyl methacrylate.

Monomers containing carboxylic acids can include, but are not limited to acrylic acid, methacrylic acid, derivatives thereof, and combinations thereof.

Monomers containing hydroxyl groups can include, but are not limited to 2-hydroxyethyl acrylate, 2-hydroxyethyl methacrylate, 2-hydroxypropyl acrylate, 4-hydroxybutyl acrylate, derivatives thereof, and combinations thereof.

In some embodiments, a copolymer provided herein comprises a ratio of about (8 to 9):(1 to 2) first monomer:second monomer. In some embodiments, the base-coat copolymer comprises a ratio of about (8 to 9):(1 to 2) THF-acrylate:APMA, and the top-coat copolymer comprises a ratio of about (8 to 9):(1 to 2) PMEA:APMA.

To prepare a polymer as described herein, the at least two monomers and an initiator can be dissolved in a solvent. In general, any solvent that dissolves the at least two monomers and the initiator can be used. Due to the disparate solubility of the alkoxyalkyl (meth)acrylate and the monomer containing an amine salt, judicious solvent selection may be required. Solvents can include, but are not limited to methanol/water, ethanol/water, isopropanol/water, dioxane/water, tetrahydrofuran/water, dimethylformamide/water, dimethylsulfoxide and/or water, and combinations thereof. With carboxylic acid and hydroxyl containing monomers, a wider range of solvents can be utilized that include, but are not limited to toluene, xylene, dimethylsulfoxide, dioxane, THF, methanol, ethanol, and dimethyl formamide.

Polymerization initiators can be used to start the polymerization of the monomers in the solution. The polymerization can be initiated by reduction-oxidation, radiation, heat, or any other method known in the art. Radiation cross-linking of the monomer solution can be achieved with ultraviolet light or visible light with suitable initiators or ionizing radiation (e.g. electron beam or gamma ray) without initiators. Polymerization can be achieved by application of heat, either by conventionally heating the solution using a heat source such as a heating well, or by application of infrared light to the monomer solution.

In some embodiments, the polymerization initiator is azobisisobutyronitrile (AIBN) or a water soluble AIBN derivatives (2,2′-azobis(2-methylpropionamidine) dihydrochloride), or 4,4′-azobis(4-cyanopentanoic acid). Other useful initiators include N,N,N′,N′-tetramethylethylenediamine, ammonium persulfate, benzoyl peroxides, and combinations thereof, including azobisisobutyronitriles. Initiator concentrations can range from 0.25% to 2% w/w of the mass of the monomers in solution. The polymerization reaction can be performed at elevated temperatures, such as in the range from about 65 to about 85° C. After polymerization is completed, the polymer can be recovered by precipitation in a non-solvent and dried under vacuum.

In some embodiments, the copolymer comprises:

poly(2-methoxyethyl acrylate)-co-poly(hydroxybutyl acrylate);

poly(2-methoxyethyl acrylate)-co-poly[(3-aminopropyl) methacrylamide]; or

poly(2-methoxyethyl acrylate)-co-poly[(3-aminopropyl) methacrylate].

poly(tetrahydrofurfuryl acrylate)-co-poly(hydroxybutyl acrylate);

poly(tetrahydrofurfuryl methacrylate)-co-poly(hydroxybutyl acrylate);

poly(tetrahydrofurfuryl acrylate)-co-poly[(3-aminopropyl) methacrylamide];

poly(tetrahydrofurfuryl methacrylate)-co-poly[(3-aminopropyl) methacrylamide];

poly(tetrahydrofurfuryl acrylate)-co-poly[(3-aminopropyl) methacrylate]; or

poly(tetrahydrofurfuryl methacrylate)-co-poly[(3-aminopropyl) methacrylate].

Following polymerization, reactive groups, such as acrylates and/or methacrylates, can be added to the copolymer via the hydroxyl, amine, and/or carboxylic acid groups of the at least one second monomer. In general, the derivatization compound can be a hetero-bifunctional compound. One moiety reacts with the hydroxyl, amine, and/or carboxylic acid groups of the copolymer. The other moiety can be an acrylate or methacrylate group. Suitable derivatization compounds include 2-isocyanatoethyl acrylate, 2-isocyanatoethyl methacrylate, N-hydroxysuccinimide ester of acrylic acid, N-hydroxysuccinimide ester of methacrylic acid, hetero-bifunctional poly(ethylene glycol) with acrylate and isocyanate groups, combinations thereof, and derivatives thereof.

To prepare the derivatized copolymer, the copolymer, derivatization compound, and any catalyst can be dissolved in a solvent. In general, any solvent that dissolves the two or more monomers and the initiator can be used. Preferred solvents include dimethyl formamide, dimethyl sulfoxide, toluene, acetone, acetonitrile, N,N-dimethylacetamide, N-methyl-2-pyrrolidone, and combinations thereof.

When reacting a derivatization with a nucleophilic group of the base coat copolymer, the molar equivalent of derivatization agent can range from about 5% to about 80% of the available nucleophilic groups, or from about 10% to about 50%. This level of derivatization corresponds to a range of about 4 to about 50 reactive groups per molecule. Additionally, the addition of a Lewis base such as a catalyst can be used. Lewis bases can include triethylamine and pyridine, such as in a concentration of about 1% to about 10% of the moles of the derivatization compound added. The reaction proceeds at ambient or elevated temperature, preferably 45° C. After the derivatization is complete, the completed, decorated copolymer can be recovered by precipitation in a non-solvent and dried under vacuum.

After the copolymer is synthesized, it can be incorporated into a coating solution. The coating solution can include a solvent, copolymer, initiator and optionally a surfactant. In general, any solvent or mixtures of solvents may be utilized, provided that the components can be dissolved into the solvent or solvent mixtures. Suitable solvents include water, alcohols, glycol ethers, aromatics, polar aprotic solvents, and combinations thereof. In some embodiments, solvents can include methanol, ethanol, isopropyl alcohol, 2-ethoxy ethanol, propylene glycol monomethyl ether acetate, benzene, toluene, xylene, dimethyl formamide, dimethyl sulfoxide, and combinations thereof. The copolymer is dissolved into the selected solvent at a concentration ranging from 0.05% w/w to 35% w/w or more, depending on the desired viscosity of the basecoat solution.

Initiators can include Norrish Type I initiators, Norrish Type II initiators, and combinations thereof. The initiator concentration in the solvent ranges from about 0.1% to about 6%, preferably about 0.5%. Examples of suitable Norrish Type I or free-radical photo-initiators are benzoin derivatives, methylolbenzoin and 4-benzoyl-1,3-dioxolane derivatives, benzilketals, α,α-dialkoxyacetophenones, α-hydroxy alkylphenones, α-aminoalkylphenones, acylphosphine oxides, bisacylphosphine oxides, acylphosphine sulphides, halogenated acetophenone derivatives, and the like. Commercial examples of suitable Norrish Type I photoinitiators are Irgacure 2959 (2-hydroxy-4′-(2-hydroxyethoxy)-2-methyl propiophenone), Irgacure 651 (benzildimethyl ketal or 2,2-dimethoxy-1,2-diphenylethanone) (Ciba-Geigy), Irgacure 184 (1-hydroxy-cyclohexyl-phenyl ketone as the active component) (Ciba-Geigy), Darocur 1173 (2-hydroxy-2-methyl-1-phenylpropan-1-one as the active component) (Ciba-Geigy), Irgacure 907 (2-methyl-1-[4-(methylthio)phenyl]-2-morpholino propan-1-one) (Ciba-Geigy), Irgacure 369 (2-benzyl-2-dimethylamino-1-(4-morpholinophenyl)-butan-1-one as the active component) (Ciba-Geigy), Esacure KIP 150 (poly {2-hydroxy-2-methyl-1-[4-(1-methylvinyl)phenyl]propan-1-one}) (Fratelli Lamberti), Esacure KIP 100 F (blend of poly {2-hydroxy-2-methyl-1-[4-(1-methylvinyl)phenyl]propan-1-one} and 2-hydroxy-2-methyl-1-phenyl-propan-1-one) (Fratelli Lamberti), Esacure KTO 46 (blend of poly {2-hydroxy-2-methyl-1-[4-(1-methylvinyl)phenyl]propan-1-one}, 2,4,6-trimethylbenzoyldiphenyl-phosphine oxide, and methylbenzophenone derivatives) (Fratelli Lamberti), acylphosphine oxides such as Lucirin TPO (2,4,6-trimethylbenzoyl diphenyl phosphine oxide) (BASF), Irgacure 819 (bis(2,4,6-trimethylbenzoyl)-phenyl-phosphine-oxide) (Ciba-Geigy), Irgacure 1700 (25:75% blend of bis(2,6-dimethoxybenzoyl)2,4,4-trimethyl-pentyl phosphine oxide and 2-hydroxy-2-methyl-1-phenyl-propan-1-one) (Ciba-Geigy), and the like. Also, mixtures of type I photo-initiators can be used.

Examples of Norrish Type II photo-initiators that can be used in the coating formulation described herein can include aromatic ketones such as benzophenone, xanthone, derivatives of benzophenone (e.g. chlorobenzophenone), blends of benzophenone and benzophenone derivatives (e.g. Photocure 81, a 50/50 blend of 4-methyl-benzophenone and benzophenone), Michler's Ketone, Ethyl Michler's Ketone, thioxanthone and other xanthone derivatives like Quantacure ITX (isopropyl thioxanthone), benzil, anthraquinones (e.g. 2-ethyl anthraquinone), coumarin, or chemical derivatives or combinations of these photoinitiators.

The coating solution may also contain a surfactant. In general, any surfactant may be used. However, surfactants can include sodium lauryl sulfate, Tween 20, Span 80, Triton X-100, Pluronic F68, Pluronic L-81, combinations thereof, and derivatives thereof. The optional surfactant can be dissolved into the selected solvent at a concentration ranging from about 0.05% w/w to about 15% w/w.

In some embodiments, for polymeric surfaces, the surface can be coated in three steps: cleaning, coating, and washing. To clean the substrate, it is first cleaned by a solvent wipe to remove any gross contamination from its surface. In general, any solvent can be used if it does not dissolve or degrade the substrate. Such solvents can include methanol, ethanol, propanol, butanol, N,N′-dimethylformamide, dimethyl sulfoxide, diglyme, triglyme, tetraglyme, tetrahydrofuran, toluene, benzene, diethyl ether, methyl tert-butyl ether, acetone, hexanes, dichloroethane, dichloromethane, water, and combinations thereof. In one embodiment, a combination of ethanol/water 1:1 (v/v) can be used.

Following solvent cleaning, the substrate may be plasma treated to further clean its surface. Plasmas derived from various gases can be used, but in some embodiments, gases can include argon and oxygen. In some embodiments, both argon and oxygen plasmas may be utilized. With the substrate suitability cleaned, it is ready to be coated.

The coating solution may be applied to the substrate by dip coating, spraying, brushing, and combinations thereof. In one embodiment, dip coating is used for application of the coating solution. For example, the substrate may be immersed in a polymer solution for about 5 to about 600 seconds. In one embodiment, duration is about 120 seconds. After the incubation, the substrate can be removed from the coating solution and irradiated with UV light with a wavelength ranging from about 10 nm to about 400 nm. Combinations of wavelengths in this range can also provide a suitably cured substrate. Irradiation wavelengths can include about 254 nm and about 365 nm. The UV irradiation time can range from about 0.1 min to about 10 min. The coating process is complete after the UV irradiation of the substrate.

Finally, the substrate may optionally be rinsed in methanol, ethanol, isopropanol, butanol, toluene, water, N,N′-dimethylformamide, dimethyl sulfoxide, diethyl ether, methyl tert-butyl ether, ethyl acetate, chloroform, and combinations thereof. After rinsing, the substrate may be dried using heat or vacuum. The substrate may be heated at temperatures ranging from about 40 to about 100° C., with or without vacuum. In one embodiment, drying conditions are 40° C. under vacuum. At this point, the substrate is ready for packaging.

In some embodiments, a second coating, a top-coat, of a copolymer described herein, may be applied to the coated substrate, which initial coating may be referred to as a base-coat. In some embodiments, the base-coat copolymer and the top-coat copolymer are covalently linked.

In some embodiments, the copolymer is a top-coat copolymer and the at least one copolymer further comprises a base-coat copolymer, the top-coat copolymer is covalently linked to the surface via covalent linkage to the base-coat copolymer, and the base-coat copolymer is covalently linked to the surface.

In some embodiments, the base-coat copolymer is prepared from the free radical polymerization of:

tetrahydrofurfuryl acrylate or tetrahydrofurfuryl methacrylate; and

at least one monomer containing an amine, a carboxylic acid, or a hydroxyl functionality.

In some embodiments, the top-coat copolymer comprises:

poly(2-methoxyethyl acrylate)-co-poly(hydroxybutyl acrylate);

poly(2-methoxyethyl acrylate)-co-poly[(3-aminopropyl) methacrylamide]; or

poly(2-methoxyethyl acrylate)-co-poly[(3-aminopropyl) methacrylate].

In some embodiments, the base-coat copolymer comprises:

poly(tetrahydrofurfuryl acrylate)-co-poly(hydroxybutyl acrylate);

poly(tetrahydrofurfuryl methacrylate)-co-poly(hydroxybutyl acrylate);

poly(tetrahydrofurfuryl acrylate)-co-poly[(3-aminopropyl) methacrylamide];

poly(tetrahydrofurfuryl methacrylate)-co-poly[(3-aminopropyl) methacrylamide];

poly(tetrahydrofurfuryl acrylate)-co-poly[(3-aminopropyl) methacrylate]; or

poly(tetrahydrofurfuryl methacrylate)-co-poly[(3-aminopropyl) methacrylate].

In some embodiments, the base-coat copolymer comprises poly(tetrahydrofurfuryl acrylate)-co-poly[(3-aminopropyl) methacrylamide] or poly(tetrahydrofurfuryl acrylate)-co-poly[(3-aminopropyl) methacrylate], and the top-coat copolymer comprises poly(2-methoxyethyl acrylate)-co-poly[(3-aminopropyl) methacrylamide] or poly(2-methoxyethyl acrylate)-co-poly[(3-aminopropyl) methacrylate].

In some embodiments, the base-coat copolymer and the top-coat copolymer are further modified to contain a plurality of reactive moieties. In some embodiments, the plurality of reactive moieties comprises a plurality of acrylates.

In some embodiments, provided herein are coated substrates comprising a coating comprising the base-coat copolymer and top-coat copolymer described herein.

In some embodiments, provided herein are medical devices comprising the surface-treated substrates described herein, or coated substrates as described herein.

In some embodiments, the medical devices described herein comprise a syringe a catheter, a probe, or a combination thereof. In some embodiments, the catheter is a microcatheter.

In some embodiments, the medical devices described herein comprise an implantable medical device. In some embodiments, the implantable medical device comprises a flat coupon, a hypo tube, a wire, a woven wire, a laser cut object, or a combination thereof.

### Example 1

### Preparation of Copolymer of Poly(2-Methoxyethyl Acrylate)-co-Poly[(3-Aminopropyl) Methacrylamide Hydrochloride]

To a mixture of 40 mL water and 40 mL methanol, 40 g of 2-methoxyethylacrylate, 4 grams of 3-aminopropyl methacrylamide hydrochloride, and 440 mg of [4,4′-azobis(4-cyanovaleric acid)] are dissolved. Polymerization occurs over 4 hours at 80° C. The copolymer is recovered by precipitation in a mixture of isopropanol: hexanes (500 mL: 500 mL). The copolymer is re-dissolved in a mixture of 80 mL tetrahydrofuran and 20 mL ethanol and reprecipitated in a mixture of isopropanol hexanes (400 mL: 600 mL). The copolymer is re-dissolved in a mixture of 80 mL tetrahydrofuran and 20 mL ethanol and reprecipitated in a mixture of isopropanol: hexanes (300 mL: 700 mL). Finally, the copolymer is stirred in 1 L of hexane for 1 hour and dried under vacuum. The copolymer is a white, foamy solid.

The resulting polymer is dissolved in N,N′-dimethylformamide (91 mL). To this solution is added 4-methoxyphenol (91 mg) and 2-isocyanatoethyl acrylate (1.82 mL). The solution is stirred from 5 hours to overnight. The solution is the poured over isopropanol: hexanes mixture (700 mL, 50/50, v/v) to precipitate the polymer. The precipitated polymer is dissolved in ethanol (80 mL, containing 80 mg of 4-methoxyphenol) and precipitated in isopropanol: hexanes mixture (700 mL, 40/60, v/v). In the third precipitation, the precipitated polymer is dissolved in ethanol (80 mL, containing 80 mg of 4-methoxyphenol) and precipitated in isopropanol: hexanes (700 mL, 20/80, v/v). The polymer is dried under reduced pressure overnight, resulting in an off-white, foamy solid.

### Example 2

### Preparation of Copolymer of Poly(Tetrahydrofuryl Acrylate)-co-Poly[(3-Aminopropyl) Methacrylamide Hydrochloride]

To a mixture of 40 mL water and 40 mL methanol, 40 g of tetrahydrofurfuryl acrylate, 4 grams of 3-aminopropyl methacrylamide hydrochloride, and 440 mg of [4,4′-azobis(4-cyanovaleric acid)] are dissolved. Polymerization occurs over 20 hours at 65° C. The copolymer is recovered by precipitation in a mixture of isopropanol/hexanes (500 mL: 500 mL). The copolymer is re-dissolved in 100 mL tetrahydrofuran and reprecipitated in a mixture of isopropanol: hexanes (400 mL: 600 mL). The copolymer is re-dissolved in 100 mL tetrahydrofuran and reprecipitated in a mixture of isopropanol hexanes (300 mL: 700 mL). Finally, the copolymer is stirred in 1 L of hexane for 1 hour and dried under vacuum. The copolymer is a slightly orange, foamy solid.

The resulting polymer is dissolved in N,N′-dimethylformamide (91 mL). To this solution is added 4-methoxyphenol (91 mg) and 2-isocyanatoethyl acrylate (1.82 mL). The solution is stirred for anytime from 5 hours to overnight. The solution is the poured over isopropanol: hexanes mixture (700 mL, 50/50, v/v) to precipitate the polymer. The precipitated polymer is dissolved in tetrahydrofuran (80 mL, containing 80 mg of 4-methoxyphenol) and precipitated in isopropanol: hexanes mixture (700 mL, 40/60, v/v). In the third precipitation, the precipitated polymer is dissolved in tetrahydrofuran (80 mL, containing 80 mg of 4-methoxyphenol) and precipitated in isopropanol: hexanes (700 mL, 20/80, v/v). The polymer is dried under reduced pressure overnight, resulting in the decorated polymer.

### Example 3

### Preparation of a Decorated Copolymer of Poly(2-Methoxyethyl Acrylate)-co-Poly[Hydroxybutyl Acrylate]

Methoxyethyl acrylate (66.7 g), 18.5 g of 4-hydroxybuyl acrylate and 250 mL of toluene are combined in 1 L round bottom flask. The solution is de-gassed by purging argon gas t for 30 min. 1.0 gram of AIBN initiator is added and the mixture is purged with argon for another 10 min. The flask is immersed in 80° C. oil bath and reflux condenser with argon inlet is attached for 16 hours under argon. The reaction is cooled down and precipitated with 1.2 L of cold methyl t-butyl ether (MTBE). The precipitated product, a viscous polymer, is collected and dried at vacuum. Typical yield is 85-95%.

The dried polymer is dissolved in dry DMF (200 ml, ˜0.5 g/mL) and treated with 0.84 ml of triethylamine and 3.0 mL of 2-isocyanatoethyl acrylate. The mixture is heated to 45° C. for 5 hrs. Subsequently, the decorated copolymer is precipitated with 1.2 L of cold MTBE, washed 2×200 mL of MTBE, and dried at high vacuum.

### Example 4

### Preparation of a Coating Solution

To prepare the coating solution, the polymer (2.98 g) from Example 3 is dissolved in ethanol (80 g), N,N′-dimethylformamide (10 mL), and water (46.7 mL). Benzophenone (454 mg) and 1-hydroxycyclohexyl phenyl ketone (468 mg) are added to the solution. The prepared coating solution is stored in an amber bottle at 4° C. or below until use.

### Example 5

UV Irradiation of a PVC Substrate

PVC film treated with plasma is dipped for 120 sec in the coating solution from Example 4 and removed to dry at ambient conditions for 1.5-2 hours. The films are further dried under reduced pressure from 2 hours to overnight. The films are placed in a Harland PCX UV Coating Machine and irradiated for 30 seconds on each side. The power of the UV lamps is 2.2-2.5 milliwatts/cm² and the wavelength 335 nm.

### Example 6

UV Irradiation of a PVC Substrate

PVC film treated with plasma is dipped for 120 sec in the coating solution from Example 4 and removed. The films are placed in a Harland PCX UV Coating Machine and irradiated for 30 seconds on each side. The power of the UV lamps is 2.2-2.5 milliwatts/cm² and the wavelength 335 nm.

### Example 7

Washing of a Coated PVC Substrate

The PVC films from Example 5 or Example 6 are submerged in ethanol: H₂O solution (50/50, v/v) and shaken on an orbital shaker at 200 rpm for 8 hours to overnight. The films are lifted out of the solution and rinsed sequentially with ethanol: H₂O solution (50/50, v/v) and H₂O. The rinsed films are dried at ambient conditions or under reduced pressure overnight.

### Example 8

Contact Angle Analysis of a Coated PVC Substrate

PVC film substrate surface modification is evaluated using a Rame-Hart Goniometer. PVC films with two different plasticizers, tris (2-ethylhexyl) trimellitate (TOTM) and 1,2-cyclohexane dicarboxylic acid diisononyl ester (DINCH), are evaluated. The contact angle results are shown in the table below.

| | | | | Washed | | |
|---|---|---|---|---|---|---|
| | | Dipped | Irriated | with | | |
| | | in | with | Ethanol/ | | |
| | | coating | UV | H₂O for | | |
| | Plasma | soluton | light | 8 hours | TOTM | DINCH |
| 1 | N | N | N | N | 99° | 96° |
| 2 | N | N | Y | Y | 98° | 92° |
| 3 | Y | N | Y | Y | 75° | 83° |
| 4 | Y | Y | Y | Y | 102° | 92° |

### Example 9

### Preparation of a Decorated Copolymer of Poly(Tetrahydrofuryl Acrylate)-co-Poly[(3-Aminopropyl) Methacrylamide Hydrochloride]

The copolymer of Example 2 is treated with 2-isocyanatoethyl acrylate similarly to Example 3 to prepare an acrylate-decorated copolymer of poly(tetrahydrofuryl acrylate)-co-poly[(3-aminopropyl) methacrylamide].

### Example 10

### Preparation of a Surface-Treated Substrate

An acrylate-decorated copolymer analogous to Example 9 is applied to a surface of a poly vinylchloride (PVC) substrate analogous to Examples 4 and 5 or 4 and 6, and optionally including Example 7. This provides a base-coated PVC substrate, which is further coated with a top-coat of an acrylate-decorated copolymer analogous to Example 1 to provide a surface-treated substrate comprising a PVC substrate covalently linked to a base-coat copolymer and a top-coat copolymer covalently linked to the base-coat copolymer. In some embodiments, the acrylate-decorated base-coat copolymer comprises a ratio of about (8 to 9):(1 to 2) THF-acrylate:APMA, and the acrylate-decorated top-coat copolymer comprises a ratio of about (8 to 9):(1 to 2) PMEA:APMA.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A surface-treated substrate, comprising
a substrate including a surface, and
at least one copolymer,
wherein the surface is covalently coupled to the at least one copolymer, wherein a copolymer of the at least one copolymer is prepared from the free radical polymerization of: (A) an alkoxyalkyl (meth)acrylate; and (B) at least one monomer containing an amine, a carboxylic acid, or a hydroxyl functionality.

2. The surface-treated substrate of claim 1, wherein the substrate comprises a thermoplastic polyurethane, a thermoplastic elastomer, a thermoset elastomer, a polyamide, a polyester, a polystyrene, a polyether ether ketone, a polyethylene vinyl acetate, a polyvinylidene fluoride, a polypropylene, polyethylene, a polyvinyl chloride, a polycarbonate, or a combination thereof.

3. The surface-treated substrate of claim 1, wherein the at least one copolymer is further modified to contain a plurality of reactive moieties.

4. The surface-treated substrate of claim 3, wherein the plurality of reactive moieties is a plurality of acrylates.

5. The surface-treated substrate of claim 1, wherein:
the at least one monomer containing an amine comprises 3-aminopropyl methacrylamide, 2-aminoethyl methacrylate, N-(3-methylpyridine)acrylamide, 2-(N,N-dimethylamino)ethyl methacrylate, 2-(N,N-dimethylamino)ethyl acrylate, 2-(tert-butylamino)ethyl methacrylate, methacryloyl-L-lysine, N-(2-(4-aminophenyl)ethyl)acrylamide, N-(4-aminobenzyl)acrylamide, N-(2-(4-imidazolyl)ethyl)acrylamide, aminopropyl methacrylate, or a combination thereof;
the at least one monomer containing a carboxylic acid comprises an acrylic acid, a methacrylic acid, or a combination thereof;
the at least one monomer containing a hydroxyl group comprises 2-hydroxyethyl acrylate, 2-hydroxyethyl methacrylate, 2-hydroxypropyl acrylate, 4-hydroxybutyl acrylate, or a combination thereof.

6. The surface-treated substrate of claim 1, wherein the copolymer comprises:
poly(2-methoxyethyl acrylate)-co-poly(hydroxybutyl acrylate);
poly(2-methoxyethyl acrylate)-co-poly[(3-aminopropyl) methacrylamide]; or
poly(2-methoxyethyl acrylate)-co-poly[(3-aminopropyl) methacrylate].

7. The surface-treated substrate of claim 1, wherein the copolymer is a top-coat copolymer and the at least one copolymer further comprises a base-coat copolymer, the top-coat copolymer is covalently linked to the surface via covalent linkage to the base-coat copolymer, and the base-coat copolymer is covalently linked to the surface.

8. The surface-treated substrate of claim 7, wherein the base-coat copolymer is prepared from the free radical polymerization of:
(A) tetrahydrofurfuryl acrylate or tetrahydrofurfuryl methacrylate; and
(B) at least one monomer containing an amine, a carboxylic acid, or a hydroxyl functionality.

9. The surface-treated substrate of claim 7, wherein the top-coat copolymer comprises:
poly(2-methoxyethyl acrylate)-co-poly(hydroxybutyl acrylate);
poly(2-methoxyethyl acrylate)-co-poly[(3-aminopropyl) methacrylamide]; or
poly(2-methoxyethyl acrylate)-co-poly[(3-aminopropyl) methacrylate].

10. The surface-treated substrate of claim 7, wherein the base-coat copolymer comprises:
poly(tetrahydrofurfuryl acrylate)-co-poly(hydroxybutyl acrylate);
poly(tetrahydrofurfuryl methacrylate)-co-poly(hydroxybutyl acrylate);
poly(tetrahydrofurfuryl acrylate)-co-poly[(3-aminopropyl) methacrylamide];
poly(tetrahydrofurfuryl methacrylate)-co-poly[(3-aminopropyl) methacrylamide];
poly(tetrahydrofurfuryl acrylate)-co-poly[(3-aminopropyl) methacrylate]; or
poly(tetrahydrofurfuryl methacrylate)-co-poly[(3-aminopropyl) methacrylate].

11. The surface-treated substrate of claim 9, wherein the base-coat copolymer comprises:
poly(tetrahydrofurfuryl acrylate)-co-poly(hydroxybutyl acrylate);
poly(tetrahydrofurfuryl methacrylate)-co-poly(hydroxybutyl acrylate);
poly(tetrahydrofurfuryl acrylate)-co-poly[(3-aminopropyl) methacrylamide];
poly(tetrahydrofurfuryl methacrylate)-co-poly[(3-aminopropyl) methacrylamide];
poly(tetrahydrofurfuryl acrylate)-co-poly[(3-aminopropyl) methacrylate]; or
poly(tetrahydrofurfuryl methacrylate)-co-poly[(3-aminopropyl) methacrylate].

12. The surface-treated substrate of claim 7, wherein the base-coat copolymer comprises poly(tetrahydrofurfuryl acrylate)-co-poly[(3-aminopropyl) methacrylamide] or poly(tetrahydrofurfuryl acrylate)-co-poly[(3-aminopropyl) methacrylate], and the top-coat copolymer comprises poly(2-methoxyethyl acrylate)-co-poly[(3-aminopropyl) methacrylamide] or poly(2-methoxyethyl acrylate)-co-poly[(3-aminopropyl) methacrylate].

13. The surface-treated substrate of claim 12, wherein the base-coat copolymer and the top-coat copolymer are further modified to contain a plurality of reactive moieties.

14. The surface-treated substrate of claim 13, wherein the plurality of reactive moieties comprises a plurality of acrylates.

15. The surface-treated substrate of claim 1, wherein the substrate comprises a polyvinyl chloride.

16. The surface-treated substrate of claim 15, wherein each ratio of (A):(B) is, independently, selected from about (8 to 9):(1 to 2).

17. A medical device, comprising a surface-treated substrate, comprising
a substrate including a surface, and
at least one copolymer,
wherein the surface is covalently coupled to the at least one copolymer, wherein a copolymer of the at least one copolymer is prepared from the free radical Polymerization of: (A) an alkoxyalkyl (meth)acrylate; and (B) at least one monomer containing an amine, a carboxylic acid, or a hydroxyl functionality.

18. The medical device of claim 17, wherein the medical device comprises a syringe a catheter, a probe, or a combination thereof.

19. The medical device of claim 17, wherein the medical device is an implantable medical device.

20. The medical device of claim 19, wherein the implantable medical device comprises a flat coupon, a hypo tube, a wire, a woven wire, a laser cut object, or a combination thereof.
